(19) United States
(12) Patent Application Publication
(10) Pub. No.: US 2006/0084068 A1
(43) Pub. Date: Apr. 20, 2006

(54) Process for detecting a nucleic acid target

(76) Inventor: Mark K. Johnson, New Berlin, WI (US)

Correspondence Address: MARK K. JOHNSON, P.O. BOX 510644, NEW BERLIN, WI, 53151, US

(21) Appl. No.: 10/960,850

(22) Filed: Oct. 7, 2004

(51) Int. Cl. C12Q 1/68; C12P 19/34

(52) U.S. Cl. 435/6; 435/91.2

(57) ABSTRACT

Described is a process for detecting the presence or absence of one or more target nucleotides. A primer is added to a solution containing DNA. The solution is put under conditions favorable to PCR amplification using sequence specific primers. Product is then measured to determine the presence or absence of target.

### FIELD

The present invention relates to a method and kit for detecting the presence or absence of a target nucleotide. The process is of particular interest in the testing of DNA samples for mutations, deletions and polymorphisms, or occurrences to the genome not inherited, such as environmentally induced mutations, deletions, substitutions and additions, and provides a general method for detecting point mutations. It is also useful in the detection and typing of infectious pathogens by analysis of their DNA.

### BACKGROUND

Several hundred genetic diseases are known to exist in man, which result from particular mutations at the DNA level. The molecular basis for certain of these diseases is already known and research is rapidly revealing the molecular basis for those genetic diseases for which the nature of the mutation is at present unknown.

The introduction of the polymerase chain reaction (PCR), as described in U.S. Pat. No. 4,683,202, issued Jul. 28, 1987 has revolutionized molecular detection of disease. With the PCR, analysis of test samples can be focused entirely on the segments of genomic DNA containing a mutation, deletion and/or polymorphism (target). Mimicking the process of DNA replication, oligonucleotides bind to complementary regions and “prime” DNA strand synthesis by a DNA polymerase. Cis-positioned primers limit the size of the segment that is produced. The reaction is repeated many times to generate large quantities of a particular segment of genomic DNA. The amplified material, subjected to the restriction enzyme, was in sufficient quantity to allow the resulting fragments to be visualized directly in a gel following electrophoresis and staining with dyes.

A much utilized method for detection of amplification product without prior separation of primer and product is the 5′ nuclease PCR assay (also referred to as the TaqMan.RTM. assay) (Holland et al., 1991, Proc. Natl. Acad. Sci. USA 88: 7276-7280; Lee et al., 1993, Nucleic Acids Res. 21: 3761-3766). This assay detects the accumulation of a specific PCR product by hybridization and cleavage of a doubly labeled fluorogenic probe (the “TaqMan” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

An alternative strategy incorporates specificity into the amplification reaction by using sequence-specific primers. This approach capitalizes on the lack of a 3′ editing function in Taq polymerase; the DNA polymerase most often used in the PCR. The absence of this enzymatic function enables a nucleotide mismatch at or near the 3′ end of a primer to prevent amplification of that primer, and the failure to form a PCR product. In this approach, primers are designed to terminate at or near the site of a known polymorphism with the ultimate base being distinctive for either the wild type or mutant base. The primers are said to be sequence-specific (SSP) or allele-specific (ASP). Following PCR, the presence or absence of an amplification product indicates the presence or absence of an allele in the genomic sample. However, the disruptive nature of a base mismatch does not ensure that an amplification product will not be formed. Consequently, subjective interpretation of signal intensity may be required when analyzing results and assigning genotype to a test sample.

PCR products from SSP reactions may be measured by fluorescent means, such as TaqMan, or by more traditional methods, such as size-fractionation by gel electrophoresis and visualization in the gel. As described, the gel endpoint is amenable to identifying multiple PCR products formed in a single reaction. In practice, constraining primer position and sequence to obtain a product of a specific size can negatively impact the yield of product, fragment visualization, and, subsequently, genotype assignment.

The present invention addresses some of the shortcomings of known methods and provides a new process for detecting DNA targets.

### SUMMARY

The SSP reaction described in the Background consists of selecting the nucleotide sequence of an oligonucleotide primer appropriately to selectively achieve primer extension of a sequence containing a target or to prevent or subdue such primer extension. In reality, preventing or subduing extension is extremely difficult and time consuming and many times cannot be done. In fact, in a diagnostic kit manufacturing setting, it is highly desirable to have numerous SSP primers amplify using the same PCR conditions and yielding the same results. This has been virtually impossible.

However, provided in this specification is a preferred embodiment for a method of detecting the presence or absence of at least one target nucleotide in one or more nucleic acids contained in a sample by treating the sample with appropriate nucleoside triphosphates, a polymerase an SSP primer specific for a target sequence and a blocking oligonucleotide. The nucleotide sequence of the detection primer being such that it is substantially complementary to the target, whereby an extension product of the detection primer is synthesized when the detection primer is complementary to the corresponding nucleotide in the target sequence and no or less extension product is synthesized when the detection primer is not complementary to the corresponding target sequence; and determining the presence or absence of the target from detecting the extension product.

While the method of the present invention is of particular interest in detecting the presence or absence of at least one specific nucleotide (e.g. mutations, polymorphisms, deletions etc.) in a preferred embodiment. For example, using the method described, one may choose to detect a DNA sequence that is different by at least one base from a known wild type sequence. The difference could be a deletion of one or more nucleotide bases, substituted bases, or even bases added to the genomic sequence to be detected. The difference may be attributable to an inherited mutation, deletion, substitution, addition, or polymorphism or it may be attributable to incidents to the genome other than genetic inheritance, such as a change of one or more bases to a known genomic sequence or foreign DNA incorporated into a cell.

In another preferred embodiment a kit for testing DNA for at least one target nucleotide, whether inherited or not inherited, comprising: a receptacle containing a primer having a nucleotide sequence substantially complementary to a sequence of the DNA and a receptacle containing a reporter.

### DETAILED DESCRIPTION

The term “nucleoside triphosphate” is used to refer to nucleosides present in either DNA or RNA and thus includes nucleosides which incorporate adenine (A), cytosine (C), guanine (G), thymine (T) and uracil (U) as base, the sugar moiety being deoxyribose or ribose. In general deoxyribonucleosides will be employed in combination with a DNA polymerase. However, other modified bases capable of base pairing with one of the conventional bases adenine, cytosine, guanine, thymine and uracil may be employed. If desired one or more of the nucleoside triphosphates present in the reaction mixture for the purpose of incorporation in to the extended primer(s) may be labeled or marked in any convenient manner.

The term “nucleotide” as used can refer to nucleotides present in either DNA or RNA and thus includes nucleotides which incorporate adenine, cytosine, guanine, thymine and uracil as base, the sugar moiety being deoxyribose or ribose. It will be appreciated however that other modified bases capable of base pairing with one of the conventional bases, adenine, cytosine, guanine, thymine and uracil, may be used in the detection primer employed in the present invention.

The enzyme for polymerization of the nucleoside triphosphates may be any compound or system which will function to accomplish the synthesis of primer extension products, including enzymes. Suitable enzymes for this purpose include, for example, E. coli DNA Polymerase I, Klenow fragment of E. coli DNA polymerase I, T4 DNA polymerase, other available DNA polymerases, reverse transcriptase, and other enzymes, including thermostable enzymes such as Taq polymerase. The term “thermostable enzyme” refers to an enzyme which is stable to heat and is heat resistant and catalyzes (facilitates) combination of the nucleotides in the proper manner to form the primer extension products which are complementary to each nucleic acid strand. Generally, the synthesis will be initiated at the 5′ end of each primer and will proceed in the 3′ direction along the template strand, until synthesis terminates, producing molecules of different lengths. There may be thermostable enzymes for example which initiate synthesis at the 3′ end and proceed in the other direction, using the same process as described above.

The expression “target” means that portion of a DNA sequence which contains at least one nucleotide of interest, whether normal, a deletion, addition, substitution, polymorphism or other; the presence or absence of which is being detected by the described process. Generally one of possibly a plurality of potential target nucleotides will be a pairing base on the genomic strand opposite the 3′-terminal end of the primer extension sequence since, in a preferred embodiment, primer extension products will be initiated at the 5′ end of each primer as described above. The 3′-terminal end may include one or more 3′ bases in the primer. Where, however, an enzyme for polymerization is to be used which initiates synthesis at the 3′ end of the detection primer and proceeds in the 5′ direction along the template strand until synthesis terminates the appropriate sequence will contain the target near or at its 5′ end.

The term “oligonucleotide” as used herein is defined as a molecule comprised of two or more deoxyribonucleotides or ribonucleotides, preferably more than three. Its exact size will depend on many factors and the exact sequence of the oligonucleotide may also depend on a number of factors as described.

The term “primer” as used herein refers to an oligonucleotide, whether occurring naturally or produced synthetically, which is capable of acting as a point of initiation of synthesis when placed under conditions in which synthesis of a primer extension product which is complementary to a nucleic acid strand is induced, i.e., in the presence of appropriate nucleoside triphosphates and an enzyme for polymerization such as DNA polymerase. An appropriate buffer (“buffer” includes pH, ionic strength, cofactors, etc.) may be used at a suitable temperature.

The primer is preferably single stranded for maximum efficiency in extension, but alternatively may be double stranded. If double stranded, the primer is first treated to separate its strands before being used to prepare extension products. Preferably, the primer is an oligodeoxyribonucleotide. The primer must be sufficiently long to prime the synthesis of extension products in the presence of the enzyme for polymerization. The exact lengths of the primers will depend on many factors, including temperature and source of primer and use of the method. For example, depending on the complexity of the target sequence, the detection primers typically contain 12-40 nucleotides, although they may contain more or fewer nucleotides. Short primer molecules generally require lower temperatures to form sufficiently stable hybrid complexes with the template.

The term “blocker” defines an oligonucleotide that has a base upstream from its own 3′ end that competes with the primer 3′ base for the same base position on a complementary genomic DNA template sample. The blocker is designed to anneal to the genomic DNA sample, downstream of the primer—except that a base upstream from its own 3′ end is in the same position, relative to the genomic DNA template sample, as the primer 3′ base. In effect a structure is presented to the polymerase wherein a primer 3′ base is not complementary to its opposing template base, yet, a base on an adjacent oligonucleotide is complementary to the template base. This structure effectively impedes the non-complementary primer 3′ base from initiating an extension product along the template. In the absence of the blocker oligonucleotide, the primer will allow the polymerase to make an extension product complementary to the template under any conditions and especially under the PCR conditions used in this specification.

The term “complementary to” is used herein in relation to nucleotides to mean a nucleotide which will base pair with another specific nucleotide. Thus adenosine triphosphate is complementary to uridine triphosphate or thymidine triphosphate and guanosine triphosphate is complementary to cytidine triphosphate. It is appreciated that while thymidine triphosphate and guanosine triphosphate may base pair under certain circumstances they are not regarded as complementary for the purposes of this specification. It will also be appreciated that while cytosine triphosphate and adenosine triphosphate may base pair under certain circumstances they are not regarded as complementary for the purposes of this specification. The same applies to cytosine triphosphate and uracil triphosphate.

The primers herein are selected to be substantially complementary to the different strands of each specific sequence to be used as a template. This means that the primers must be sufficiently complementary to hybridize with their respective strands. Therefore, the primer sequence need not reflect the exact sequence of the template. For example, where the primer comprises a nucleotide sequence complementary to the target a non-complementary nucleotide sequence may be attached to the 5′-end of the primer. The primers may utilize non-complementary nucleotides at a predetermined primer 3′ end to regulate efficiency of extension.

In a preferred embodiment, a sample of DNA possibly containing a target sequence is tested. The DNA may be double or single stranded. Typically the DNA will be obtained from an organism. Preferably membrane material is disrupted to allow primers access to the DNA.

A primer specific for a target sequence and a blocker is added to a solution containing the DNA along with an extension polymerase and dNTP's for extension. No label is required at this point, however, dNTP's may be labeled by using radioactive isotopes to label DNA fragments. Alternatively, dNTP's associated with fluorescent dyes may be used as labels that fluoresce when excited, usually when exposed to light of a certain wavelength. The DNA is then put into a condition which is amenable to extension of the primer by the polymerase (e.g. denaturing double-stranded DNA, changing heat conditions, adjusting pH etc.). The primer is efficiently extended (by appropriate means including adding a polymerase and adjusting the temperature) when the target is present. The primer with blocker is designed to efficiently extend along the DNA template potentially containing target when the target is present. However, the primer and blocker is designed to prevent extension or allow inefficient extension when the target is not present. Detection of amounts of efficient primer extension product compared with detection of amounts of inefficient primer extension product determines if target is present. Efficient primer extension will yield a greater amount of double-stranded product than inefficient primer extension. Means of detection include measuring the emissions from labels associated with extension product.

In a preferred embodiment, a sample of cells is collected from a subject to be tested, such as by a buccal scrape containing cells or a blood sample. Any appropriate collection tool may be utilized which will collect a sufficient number of cells. Any known means may be used to allow the primer to associate with the genomic DNA such as disrupting the cell membrane or lysing the cell.

A target specific primer is added to a solution containing the genomic DNA that will substantially hybridize to a target sequence located on the genomic DNA. The primer is designed with one 3′ base that is either complementary to its opposing base or not depending upon the genomic DNA. A blocker is also added to the solution. The blocker is an oligo that is complementary to the genomic DNA, 3′ to the target specific primer. It overlaps the target specific primer by one base such that the blocker base is in the same sequential position as the 3′ target specific primer base. However, the blocker base will not be the same as the 3′ target specific primer base.

For example: a genomic DNA sample to be tested contains a polymorphism at the 25^{th} genomic base position. The target specific primer contains a 3′ base at the 25^{th} position, however, in this example, it is not complementary to the polymorphism. The blocker contains a blocking base at the 25^{th} genomic base position, complementary to the polymorphism. Therefore, the blocker competitively blocks the extension of the target specific primer. Subsequently, at the indicated thermal cycling profiles, no or very little non-specific extension product is made.

The great advantage of using the blocker with the target specific primer is that, except for the 3′ base in negative samples, the primer sequence can be absolutely complementary to the genomic target sequence. In the prior art, base substitutions in SSP primers are usually used to modulate the extension efficiency of a non-annealing 3′ base.

Sequence specific primer systems in the prior art require time consuming trial and error primer design using base mismatches at positions 5′ to the 3′ end-base. Many times a SSP cannot be identified. However, using the described process, there is little trial and error and adjustments are made by lengthening or shortening the primer. It is clear that as the genome is unraveled, additional new detection products will be easily and, more importantly, more quickly be made available to US research and commerce.

Although any method of detecting PCR product may be used, two processes are particularly preferred. The first process is to detect the product by gel electrophoresis. Preferably, an E-GEL, which is poured by the manufacturer and includes ethidium bromide, is used because of its ease and quickness. When more than one sequence is being detected, the primers will be designed so that each product is a different length and may be easily distinguished in the gel.

The second preferred method, is attaching a fluorescent label. Typically the label is attached to the 5′ end of the primer. An oligo, complimentary to the primer is placed into solution with the primer. The oligo is not extendable and contains a 3′ quencher to quench the primer fluorophore. The 3′ end of the oligo is positioned in close proximity to the fluoro label when annealed. However, the melting temperature of the oligo is less than the melting temperature of the primer so that is does not interfere with PCR. The molarity of oligo should be the same as or more than the primer molarity. After PCR, the temperature of the thermal cycler should be maintained at a temperature near the melting temperature of the quenching oligo for at least one minute, to insure that the oligo anneals to the primer.

Of course, if the primer is used in an extension product, the quenching oligo will not be able to anneal to it and the primer will be unquenched. The fluorescence can then be measured. If the fluorescence is measured to be statistically greater than a control of fluorescently labeled primer in solution with quencher labeled oligo, PCR product must have formed, which is indicative of a 3′ primer base complementary to the targeted polymorphism. The fluorescence may be measured in any fluorometer including a fluorescent plate reader.

In a preferred embodiment, extension of the primer is detectable by adding a label that provides a signal when it encounters double-stranded DNA. Examples of such a labels are ethidium bromide, acridene orange, Sybr Green and Pico Green from Molecular Probes, Inc., Eugene Oreg. The signal is measured by an appropriate means and target is detected. Pico Green is an example of a fluorescence emitting label. Pico Green intercalates with double-stranded DNA and emits a fluorescence light at a defined wavelength. When the label is excited, the emitted light is detectable as indicative of double stranded DNA. Additionally, a label that indicates whether single or double stranded DNA is present may be used since in a preferred embodiment, the amount of DNA is detected.

In another preferred embodiment, extension of the primer is detectable by using fluorescently labeled dNTP's. If efficient extension of the primer occurs, the labeled dNTP's will be incorporated into double-stranded DNA. The unincorporated dNTP's may be removed by means known to the art such as a spin column designed to collect small particles (such as dNTP's) yet allowing larger particles such as DNA to pass through. The labeled dNTP's incorporated into the extension product may then be caused to fluoresce and the light measured by appropriate means such as a fluorometer.

In addition, a labeled oligo can be used to detect the extension product, if made. For example, an oligo complementary to an extension product will have a fluorescent label on one end and a quencher on the other. As in FRET, if the labeled oligo is free in solution, the quenching end will be in close proximity to the fluorescence emitting end, thereby causing the fluorophore to quenching of emitted light. In contrast, if there is extension product, the labeled oligo will anneal to its complement, causing the quencher and the fluorophore to be apart wherein fluorescence is quenched less than non-annealed oligo. The difference in emitted fluorescence between the non-annealed oligo and the annealed oligo can be measured and one can determine whether or not extension product has been made.

Alternatively, a molecular beacon can be used in the example described in the previous paragraph as the detection oligo.

The same process can be applied to any sample of DNA. The source of the DNA does not appear to be important for the processes described. An important part of this specification is the ability to detect a target that may be as small as one nucleotide. A primer's efficient extension compared to no extension or inefficient extension is all that is required and usable results may be obtained from purified or non-purified starting material including DNA contained in whole blood, lymphocytes, other cells, viruses, and bacteria.

In another preferred embodiment, genomic DNA is obtained from whole blood. The DNA may be purified using a purification method known to the art such as the Qiagen system. However, other purification systems are sufficient as well as no purification of the cellular DNA. The genomic DNA obtained is heated for a time sufficient to denature the double-stranded DNA. The process proceeds as previously described.

In another preferred embodiment, the target base may be a gene polymorphism. In this instance the target base is one of two choices. For example, for a specific polymorphism, perhaps 90% of the human population has the target base A but 10% of the population has the target base C. To detect the polymorphism, a primer is developed that is substantially complementary to the DNA sequence containing the polymorphism wherein the terminal base of the primer is a G which is in position to either anneal to the target base or not anneal if the target base is not present. The blocker is added which contains a T base to compete off the primer 3′ G if the sample of genomic DNA tested contains the target base A. If the genomic DNA contains the target base C, the complementary G base will anneal by competing off the blocker A base. A polymerase is used to perform a polymerase chain reaction (PCR) to extend the primers in solution with the DNA to be tested. If the target base is A, the G does not anneal and the partially annealed primer will not be efficiently extended and will not produce as much extension product as the complementary T. If the target base is C, the G will anneal and the primer will be fully annealed and it will be extended efficiently when compared to a terminal base of T. Therefore, in this example, the presence of extension product indicates the presence of target base in the sample DNA. One can manipulate the terminal bases on the primer and the blocker to obtain an extension product detecting the presence of any potential target base.

Detection methods previously described may be used to detect the presence of extension product in the prior examples. A dye (such as Molecular Probes PicoGreen or OliGreen) is added to the solution as previously described. The solution is then placed into a detector that can excite the dye to produce a detectable wavelength emission. Examples of detectors include but are not limited to flowcytometers (Beckton-Dickenson) and the Luminex 100 (Luminex Corporation). Detection of emissions indicates that an extension product is present.

In another preferred embodiment, the particle itself contains a detectable label, preferably a fluorescent signal emission. Therefore, the particle will emit a signal when appropriately excited as well as any attached fluorescently dyed extension product. In this manner, many different primers complementary to their unique portions of the sample DNA are used to produce multiple extension products, each of which will hybridize to specific oligonucleotides attached to particles. Each particle is manufactured to contain a unique signal that is specific to the individual sequence of the oligonucleotide attached. The detector can detect the unique particle signal in addition to the signal provided by the dyed extension product. Therefore, the particle is identified with extension product. If no extension product is present, a particle emission will be detectable but no extension product emission will be produced and the detector provides data indicating no extension product associated with a particular particle.

In another preferred embodiment, beads or other particles contain attached primers for producing extension product. The particles are added to a mixture of sample DNA prepared for the purpose of allowing extension product. The primers, which are unique for a particular target containing (or adjacent) sequence, anneal to their complementary sequence to either produce or not produce extension product according to previously described methods. After extension according to previously described methods, the mixture is heated to a temperature that allows the extension product and primer to disassociate from the template DNA. Particles are then detectable as well as any extension product attached. Again, multiple primers may be used as described.

In another preferred embodiment, sample DNA template containing target is placed in a solution prepared for extension product. Extension product is produced according to any of the previously described processes. After extension conditions are employed, the solution is heated to a temperature sufficient to allow disassociation of extension product, if any, from template. The solution is then added to separation process that separates genomic DNA from extension product and also removes a large percentage of remaining primers not extended (Wizard Preps, Promega Corporation). The separation process produces a solution of higher concentration extension product, if any extension product is made, relative to the solution before separation. The extension product is then dyed with a fluorescence emitting label and is detectable by known methods including flowcytometry and cytofluor detection.

In another preferred embodiment, PCR product may be detected by absorption. In this case, an added label is not required.

In another preferred embodiment, the presence or absence of at least one target nucleotide in a DNA sequence is determined. A first primer is added to a solution containing a first sample of genomic DNA. A second primer is added to a solution containing a second sample of the same genomic DNA. The second primer is substantially similar to the first primer except for one or more of the last 3 bases at the 3′ end. In most cases the last base differs between the primers. A polymerase, such as Taq polymerase, is added to each sample.

Examples of the present invention are provided for illustrative purposes and not to limit the scope of the invention.

### EXAMPLE 1

Sample Preparation

Draw several mLs of venous blood into an EDTA tube and mix thoroughly by gently inverting the tube several times. Blood may be stored at 2-8° C. for several days before processing. Extract the DNA using either published techniques or a commercially available kit. Note that the method used for DNA isolation may dictate the volume and storage conditions of the blood. Resuspend the DNA in distilled water or 10 mM Tris (pH 7.0-7.3) to a concentration of 50 to 500 ng/μL. DNA that cannot be used immediately may be held at 2-8° C. for several days. For longer-term storage, samples should be stored frozen in a constant-temperature freezer. Excess contaminating protein, heparin, or EDTA may interfere with PCR extension of the purified DNA.

### EXAMPLE 2

Compositions

Master Extension Mix contains next three (3) lines of materials
10 mM Tris (pH 8.3), 50 mM KCl, 0.01% Gelatin: Sigma [St Louis, Mo.]
200 μM dNTP(each): Pharmacia Biotech [Piscataway, N.J.]
0.5 μM primers: Custom synthesis from Genosys [The Woodlands, Texas]
Primer 1 sequence=5′ 15 bases complementary to human HPA1 section+TC T 3′
Primer 2 sequence=5′ 15 bases complementary to human HPA1 section+TC C_{3′}

The 3′ T in primer 1 is complementary to the opposing base in HPA1. However, the 3′ C is not complementary if the A is present, therefore, promoting no extension or inefficient extension. Boil 20-500 ng/μL genomic DNA for 1 minute to denature. 0.5 units/mL Taq polymerase: Perkin Elmer [Foster City, Calif.], Promega (Madison, Wis.)

Prepare 90 μL of a fresh working dilution of Taq DNA polymerase (final concentration of 0.2 U/μL) with molecular biology grade water in a microfuge tube placed on ice.

| | | |
|---|---|---|
| Number of samples: | 4 | |
| Number of controls | 4 | |
| Add two for pipetting errors | 2 | |
| Total Number of Reactions | 10 | |
| Number of reactions | 10 | |
| Amount of Taq per reaction | ×.5 | units |
| Total Units of Taq Required | 5.0 | units |
| Total units of Taq required | 5.0 | |
| Concentration of Taq stock | ÷5 | U/μL |
| Total Volume of Taq stock Required | 1 | μL. |
| Total volume required | 90 | μL |
| Volume of Taq | −1.0 | μL |
| Volume of Water Required | 89.0 | μL |
| Total number of reactions | 10 | |
| Volume of Extension Mix per reaction | ×25 | μL |
| Total Volume of Extension Mix to Add to the Diluted Taq | 250 | μL |

Add 250 μL of refrigerated Extension Mix to the microfuge tube containing the 90 μLs of freshly diluted Taq DNA polymerase and vortex briefly to mix. Final volume equals 340 μL. If not extending 8 samples, then adjust the appropriate number in the previous equation to determine the required volume.

Pipette into each extension tube (held on ice):
35 μL Taq and Extension Mix solution: 2 tubes with primer 1; 2 tubes with primer 2; 2 tubes with no primer; 2 tubes with no Taq and primer 1;
1-15 μL boiled Genomic DNA (deliver ˜50 ng of isolated DNA per tube)
0-14 μL Water to bring final reaction volume to 50 μL

Cap tubes and temporarily store on ice.

Perform extension using the following protocol. Transfer the chilled extension tubes to a heating device: in the present example a thermal cycler was utilized.

### EXAMPLE 3

Aug. 13, 2003, test 2
GENOMIC MIX

| | | | | | | | |
|---|---|---|---|---|---|---|---|
| 21.125 | μL | H₂O | × | 11 Tubes | = | 232.38 | μL |
| 2.500 | ″ | Buffer | × | ″ | = | 27.50 | ″ |
| 1.250 | ″ | Genomic | × | ″ | = | 13.75 | ″ |
| | | (100 ng) | | | | | |
| 0.125 | ″ | Taq | × | ″ | = | 1.38 | ″ |
| 25.000 | μL | | | | | 275.00 | μL |

| GENOMIC DNA | Monica | ATR | JBA | AMA310 |
|---|---|---|---|---|
| HPA1 | a/a | b/b | a/a | a/a |
| HPA2 | a/a | a/a | a/b | a/a |
| HPA3 | a/a | b/b | a/b | a/b |
| HPA4 | a/a | a/a | b/b | a/a |
| HPA5 | a/a | a/a | a/a | b/b |

Dried the five primer sets a/a and b/b in separate wells+¼HGH in each;
added above genomes;

Ran PCR;

| | | |
|---|---|---|
| 6 cycles | 26 cycles | hold |

| 94° | 57° | 72° | 94° | 65° | 72° | 4° |
|---|---|---|---|---|---|---|
| 0:01 | 0:25 | 0:10 | 0:01 | 0:25 | 0:10 | ∞ |

then, 25 min. 4% dual gel.

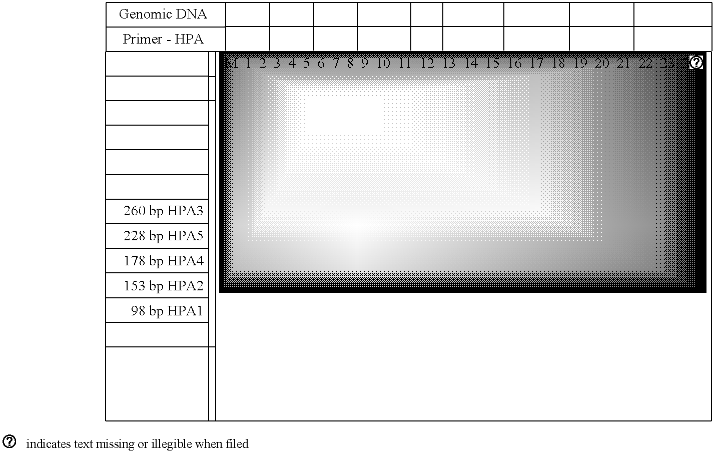

### EXAMPLE 4

Jul. 19, 2003
GENERIC MIX

| | | | | | | | |
|---|---|---|---|---|---|---|---|
| 16.125 | μL | H₂O | × | 4 Tubes | = | 64.50 | μL |
| 2.500 | ″ | Buffer | × | ″ | = | 10.00 | ″ |
| 2.500 | ″ | dNTP | × | ″ | = | 10.00 | ″ |
| 1.250 | ″ | Primer | × | ″ | = | 5.00 | ″ |
| 1.250 | ″ | Rev primer | × | ″ | = | 5.00 | ″ |
| 1.250 | ″ | Genomic | × | ″ | = | 5.00 | ″ |
| 0.125 | ″ | Taq | × | ″ | = | 0.50 | ″ |
| 25.000 | μL | | | | | 100.00 | μL |

| | | monica | anna |
|---|---|---|---|
| GENOMIC DNA | | HPA1 a/a | HPA1 b/b |
| 59.50 μL | | | |
| 10.00 μL | | | |
| 10.00 μL | | | |
| 5.00 μL | | forward primer a | forward primer a |
| | | or | or |
| | | forward primer b | forward primer b |
| 5.00 μL | | ablock | ablock |
| | | or | or |
| | | ablock | ablock |
| 5.00 μL | | Reverse primer | Reverse primer |
| 5.00 μL | genomic | | |
| 0.50 μL | Taq | | |
| 100.00 μL | Total | | |

Ran PCR;

| 6 cycles | | | 26 cycles | | | hold |
|---|---|---|---|---|---|---|
| 94° | 57° | 72° | 94° | 65° | 72° | 4° |
| 0:01 | 0:20 | 0:10 | 0:01 | 0:15 | 0:10 | ∞ |

then, 15 min. gel.

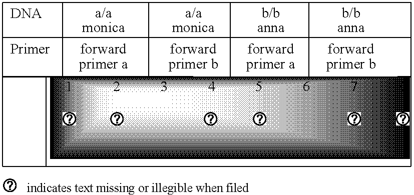

### EXAMPLE 5

Jul. 22, 2003
GENERIC MIX

| | | | | | | | |
|---|---|---|---|---|---|---|---|
| 16.125 | μL | H₂O | × | 4 Tubes | = | 64.50 | μL |
| 2.500 | ″ | Buffer | × | ″ | = | 10.00 | ″ |
| 2.500 | ″ | dNTP | × | ″ | = | 10.00 | ″ |
| 1.250 | ″ | Primer | × | ″ | = | 5.00 | ″ |
| 1.250 | ″ | Rev primer | × | ″ | = | 5.00 | ″ |
| 1.250 | ″ | Genomic | × | ″ | = | 5.00 | ″ |
| 0.125 | ″ | Taq | × | ″ | = | 0.50 | ″ |
| 25.000 | μL | | | | | 100.00 | μL |

| | | Genomic DNA | |
|---|---|---|---|
| | | Monica | MIv |
| | | HPA2 a/a | HPA2 b/b |
| 59.50 μL | H₂0 | | |
| 10.00 μL | buffer | | |
| 10.00 μL | dNTP | | |
| 5.00 μL | primer | forward primer a or | forward primer a or |
| | | forward primer b | forward primer b |
| 5.00 μL | block | ablock | ablock |
| | | or | or |
| | | bblock | bblock |
| 5.00 μL | rev | reverse primer | reverse primer |
| 5.00 μL | genomic | | |
| 0.50 μL | Taq | | |
| 100.00 μL | | | |

Ran PCR:

| 6 cycles | | | 27 cycles | | | hold |
|---|---|---|---|---|---|---|
| 94° | 57° | 72° | 94° | 65° | 72° | 4° |
| 0:01 | 0:20 | 0:10 | 0:01 | 0:15 | 0:10 | ∞ |

20 min. gel at 70 v instead of 60 v: seemed to run fine.

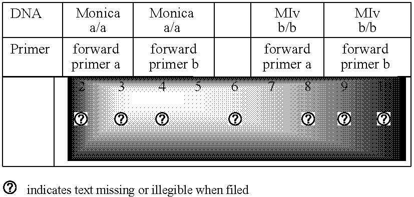

### EXAMPLE 6

Aug. 19, 2003 Test2
GENERIC MIX

| | | | | | | | |
|---|---|---|---|---|---|---|---|
| 16.125 | μL | H₂O | × | 4 Tubes | = | 64.50 | μL |
| 2.500 | ″ | Buffer | × | ″ | = | 10.00 | ″ |
| 2.500 | ″ | dNTP | × | ″ | = | 10.00 | ″ |
| 1.250 | ″ | Primer | × | ″ | = | 5.00 | ″ |
| 1.250 | ″ | Rev primer | × | ″ | = | 5.00 | ″ |
| 1.250 | ″ | Genomic | × | ″ | = | 5.00 | ″ |
| 0.125 | ″ | Taq | × | ″ | = | 0.50 | ″ |
| 25.000 | μL | | | | | 100.00 | μL |

| | Genomic DNA | | | Genomic DNA | |
|---|---|---|---|---|---|
| Reg. | Monica | ATR | Xtra | Monica | ATR |
| cycles | HPA3 a/a | HPA3 b/b | cycles | HPA3 a/a | HPA3 b/b |
| H₂0 | 54.00 μL | 66.75 μL | H₂0 | 67.50 μL | 71.90 μL |
| buffer | 10.00 μL | 10.00 μL | buffer | 10.00 μL | 10.00 μL |
| dNTP | 10.00 μL | 10.00 μL | dNTP | 10.00 μL | 10.00 μL |
| Mix | 5.00 μL | 5.00 μL | Mix | 5.00 μL | 5.00 μL |
| | (primer a | (primer a | | (primer a | (primer a |
| | or | or | | or | or |
| | primer b) | primer b) | | primer b) | primer b) |
| | and | and | | and | and |
| | Reverse | Reverse | | Reverse | Reverse |
| | primer | primer | | primer | primer |
| 3x | 20.50 μL | 7.75 μL | 1x | 7.00 μL | 2.60 μL |
| genomic | | | genomic | | |
| Taq | 0.50 μL | 0.50 μL | Taq | 0.50 μL | 0.50 μL |
| μL | 100.00 μL | 100.00 μL | μL | 100.00 μL | 100.00 μL |

Ran PCR

3x Genomic; normal cycles

| 6 cycles | | | 26 cycles | | | hold |
|---|---|---|---|---|---|---|
| 94° | 57° | 72° | 94° | 65° | 72° | 4° |
| 0:01 | 0:20 | 0:10 | 0:01 | 0:15 | 0:10 | ∞ |

1x Genomic; extra cycles

| 6 cycles | | | 30 cycles | | | hold |
|---|---|---|---|---|---|---|
| 94° | 57° | 72° | 94° | 65° | 72° | 4° |
| 0:01 | 0:20 | 0:10 | 0:01 | 0:15 | 0:10 | ∞ |

Ran 25 min. 12 channel gel

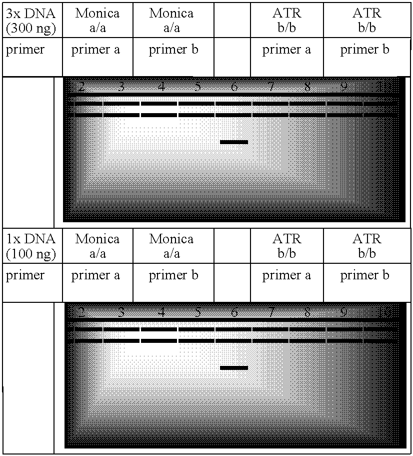

### EXAMPLE 7

Aug. 9, 2003, Test 1
GENERIC MIX

| | | | | | | | |
|---|---|---|---|---|---|---|---|
| 16.125 | μL | H₂O | X | 4 Tubes | = | 64.50 | μL |
| 2.500 | ″ | Buffer | X | ″ | = | 10.00 | ″ |
| 2.500 | ″ | dNTP | X | ″ | = | 10.00 | ″ |
| 1.250 | ″ | Primer | X | ″ | = | 5.00 | ″ |
| 1.250 | ″ | Reverse | X | ″ | = | 5.00 | ″ |
| | | primer | | | | | |
| | | primer | | | | | |
| 1.250 | ″ | Genomic | X | ″ | = | 5.00 | ″ |
| 0.125 | ″ | Taq | X | ″ | = | 0.50 | ″ |
| 25.000 | μL | | | | | 100.00 | μL |

| | | Genomic DNA | |
|---|---|---|---|
| | | monica | JBA |
| 59.50 μL | H₂0 | | |
| 10.00 μL | buffer | | |
| 10.00 μL | dNTP | | |
| 5.00 μL | primer | Primer 4a or | Primer 4a or |
| | | Primer 4b | Primer 4b |
| 5.00 μL | block | 21-4ablock | 21-4bblock |
| 5.00 μL | Reverse primer | reverse primer | reverse primer |
| 5.00 μL | genomic | HPA4 a/a | HPA4 b/b |
| 0.50 μL | Taq | | |
| 100.00 μL | | | |

Ran PCR:

| 6 cycles | | | 26 cycles | | | hold |
|---|---|---|---|---|---|---|
| 94° | 57° | 72° | 94° | 65° | 72° | 4° |
| 0:01 | 0:20 | 0:10 | 0:01 | 0:15 | 0:10 | ∞ |

20 min. gel

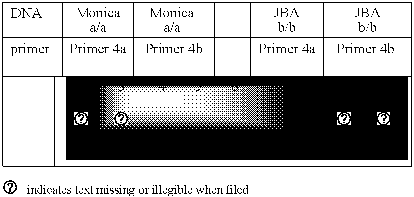

### EXAMPLE 8

Jul. 27, 2003
GENERIC MIX

| | | | | | | | |
|---|---|---|---|---|---|---|---|
| 16.125 | μL | H₂O | X | 4 Tubes | = | 64.50 | μL |
| 2.500 | ″ | Buffer | X | ″ | = | 10.00 | ″ |
| 2.500 | ″ | dNTP | X | ″ | = | 10.00 | ″ |
| 1.250 | ″ | Primer | X | ″ | = | 5.00 | ″ |
| 1.250 | ″ | Rev primer | X | ″ | = | 5.00 | ″ |
| 1.250 | ″ | Genomic | X | ″ | = | 5.00 | ″ |
| 0.125 | ″ | Taq | X | ″ | = | 0.50 | ″ |
| 25.000 | μL | | | | | 100.00 | μL |

Genomic DNA

| | | Monica | Alberta |
|---|---|---|---|
| | | HPA5 a/a | HPA5 b/b |
| 59.50 μL | H₂0 | | |
| 10.00 μL | buffer | | |
| 10.00 μL | dNTP | | |
| 5.00 μL | primer | primer 5a | primer 5a |
| | | or | or |
| | | primer 5b | primer 5b |
| 5.00 μL | block | ablock | ablock |
| | | bblock | bblock |
| 5.00 μL | Rev | reverse primer | reverse primer |
| 5.00 μL | genomic | | |
| 0.50 μL | Taq | | |
| 100.00 μL | | | |

Ran PCR;

| 6 cycles | | | 26 cycles | | | hold |
|---|---|---|---|---|---|---|
| 94° | 57° | 72° | 94° | 65° | 72° | 4° |
| 0:01 | 0:20 | 0:10 | 0:01 | 0:15 | 0:10 | ∞ |

22 min. gel @ 70V

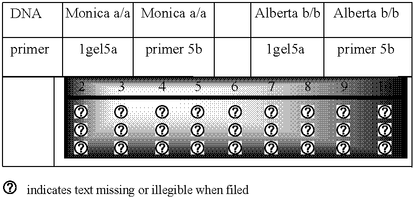

Numerous assays have been performed using different blood samples containing native genomic DNA having a known sequence. The same primers were used each time: one containing a 3′ terminal match to the HPA “a” polymorphism and one containing a 3′ terminal match to the HPA “b” polymorphism. The tests yielded similar definitive results in each case.

The foregoing is considered as illustrative only of the principles of the invention. Furthermore, since numerous modifications and changes will readily occur to those skilled in the art, it is not desired to limit the invention to the exact construction and operation shown and described. Accordingly, all suitable modifications and equivalents fall within the scope of the invention.

I claim:

1. A process for detecting the presence or absence of a target DNA sequence from a sample, comprising:
a) making a solution of a primer, a blocker, a polymerase and a sample of the target DNA sequence, wherein the target DNA sequence has at least one nucleic acid base that may change from sample to sample;
b) performing PCR amplification;
c) detecting PCR product; and,
d) determining if the nucleic acid base is present.

2. A kit for using the process of claim 1, comprising: a receptacle containing a blocker.
